SEQUENCE LISTING (6 sequences)

SEQ ID NO: 1 - protein, 280 aa, mers coronavirus
QAEGVECDFSPLLSGTPPQVYNFKRLVFTNCNYNLTKLLSLFSVNDFTCSQISPAAIASNCYSSLTLDYPLSMKSDLSVSSAGPISQFNYKQSFSNPTCLTLATCPHNLTTITKPLYSYINKCSRLLSDDRTEVPQLVNANQYSPCVSIVPSTVWEDGDYYRKQLSPLEGGGWLVASGSTVAMTEQLMGFGITVQYGTDRNSVCPKLEFANDTKIASQLGNCVEYSLYGVSGRGVFQNCTAVGVRQQRFVYDAYQNLVGYYSDDGNYYCLRACVSPVSVI

SEQ ID NO: 2 - protein, 238 aa, SARS coronavirus 2
FTVEKGIYQTSNFRVQPTESIVRFPNITNLCPFGEVFNATRFASVYAWNRKRISNCVADYSVLYNSASFSTFKCYGVSPTKLMDLCFTNVYADSFVIRCDEVRQIAPGQTGKIADYNYKLPDDFTGCVIAWNSNNLDSKVGGNYNYLYRLFRKSNLKPFERDISTEIYQAGSTPCNDVEGFNCYFPLQSYGFQPTNGVGYQPYRVVVLSFELLHAPATVCGPKKSTNLVKNKCVNFNF

SEQ ID NO: 3 - DNA, 20 bp, Artificial sequence
  Synthetic primer
gacatacttatgaggatgtc

SEQ ID NO: 4 - DNA, 22 bp, Artificial Sequence
  Synthetic primer
ctgaaagcttgacatggtgttg

SEQ ID NO: 5 - DNA, 26 bp, Artificial sequence
  Synthetic primer
gacatactgtgaggatgtcaaaaatg

SEQ ID NO: 6 - DNA, 24 bp, Artificial sequence
  Synthetic primer
ctgaaagcttgacacagtgtttgg

(19) United States
(12) Patent Application Publication
(10) Pub. No.: US 2021/0244811 A1
(43) Pub. Date: Aug. 12, 2021

(54) COMPOSITIONS IMMUNOGENIC AGAINST SARS CORONAVIRUS 2, METHODS OF MAKING, AND USING THEREOF

(71) Applicant: The University of Hong Kong, Hong Kong (CN)

(72) Inventors: Honglin Chen, Hong Kong (CN); Pui Wang, Hong Kong (CN); Zhiwei Chen, Hong Kong (CN); Kowk-Yong Yuen, Hong Kong (CN)

(21) Appl. No.: 17/172,300

(22) Filed: Feb. 10, 2021

Related U.S. Application Data
(60) Provisional application No. 62/972,616, filed on Feb. 10, 2020.
(60) Provisional application No. 63/037,645, filed on Jun. 11, 2020.

(51) Int. Cl. A61K 39/215; A61P 31/14; C12N 7/00

(52) U.S. Cl. CPC A61K 39/215; A61K 2039/5254; C12N 7/00; A61P 31/14

(57) ABSTRACT

Live attenuated viruses for protection against the novel coronavirus which emerged in Wuhan, Hubei Province of China, designated as Sars-CoV-2 by the World Health Organization (WHO) are provided. The live attenuated chimeric virus strains are based on a live attenuated influenza virus (LAIV), used a master backbone, which includes deletion of the viral virulence element, the NS1 (non-structural protein 1) (DeLNS1), engineered to express one or more antigens of the Sars-CoV-2 (herein, CoV2Ag). The chimeric virus strain is referred to generally herein, as DelNS1-Sars-CoV-2-CoV2Ag. The DelNS1-Sars-CoV-2-CoV2Ag strain preferably shows spontaneous cold adaption with preference to grow at 30-33° C. The DelNS1-Sars-CoV-2-CoV2Ag strain can be used to protect a subject in need thereof, against a challenge of Sars-CoV-2. DelNS1-Sars-CoV-2-CoV2Ag is an important strategy for making highly attenuated and immunogenic live attenuated vaccines with the ability to induce protective immunity against Sars-CoV-2.

### CROSS-REFERENCE TO RELATED APPLICATIONS

This application claims benefit of U.S. Provisional Application No. 62/972,616, filed on Feb. 10, 2020, and U.S. Provisional Application No. 63/037,645, filed on Jun. 11, 2020, which are hereby incorporated herein by reference in their entirety.

### REFERENCE TO SEQUENCE LISTING

The Sequence Listing submitted as a text file named “UHK_00924_ST25.txt,” created on May 5, 2021, 2020, and having a size of 5,769 bytes is hereby incorporated by reference pursuant to 37 C.F.R. § 1.52(e)(5).

### FIELD OF THE INVENTION

The present invention is generally in the field of live attenuated chimeric viruses containing one or antigens from Sars-CoV-2, immunogenic compositions including chimeric Sars-CoV-2 antigen containing viruses, and methods of using such compositions for inducing an immune response to Sars-CoV-2.

### BACKGROUND OF THE INVENTION

A novel coronavirus, initially designated as 2019 novel coronavirus (nCoV) by the World Health Organization (WHO), emerged in Wuhan, Hubei Province of China since December 2019. The virus has now been renamed Severe Acute Respiratory Syndrome Coronavirus 2, or Sars-CoV-2. The disease it causes is called Covid-19 (for Coronavirus Disease 2019). So far there are more than i1.3 million laboratory confirmed infections worldwide, with about 1-4% of cases fatal, depends on age and geographical locations which may have different availabilities of clinical care. The Sars-CoV-2 has disseminated globally, leading to the announcement of a pandemic caused by SAR-CoV-2 by WHO on March 12, 202. There are two possibilities of the subsequent prevalence: (1) Sars-CoV-2 will disappear from humans after a huge intervention measures currently implanted by China and many other countries; (2) Sars-CoV-2 may become a common cold virus and continue to circulate in humans, like other human coronavirus. Current situation indicates there is little possibility that SARS-CoV-2 will be disappear from humans. How humans will get along with this virus has become a reality. There are three coronaviruses that have crossed species barriers and infected human since 2002/2003 of SARS coronavirus. It is reasonably to believe that other coronavirus from animal sources may emerge and infect humans in future. A rapid responsive and effective vaccine is needed for the current ongoing pandemic caused by Sars-CoV-2 and future emerging coronavirus. Further, Humans do not have preexisting immunity to Sars-CoV-2 and there is a concern that this virus may lead to significant mobility and mortality worldwide. A vaccine for prevention of infection or alleviate morbidity or mortality caused by this Sars-CoV-2 is urgently needed

Novel strategies to develop an effective vaccine against the Sars-CoV-2 with properties to provide broad cross protective activity are necessary.

It is an object of the present invention to provide a safe and effective live attenuated coronavirus.

It is also an object of the present invention to provide methods of generating live attenuated coronavirus vaccine.

It is a further object of the present invention to provide methods of eliciting an immune response against coronavirus in a mammal.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY OF THE INVENTION

Compositions immunogenic against Sars-CoV-2, methods of making and using, are provided. The compositions include live attenuated chimeric viruses expressing one or more antigens of the Sars-CoV-2 (herein, CoV2Ag) are provided, methods of making and using thereof, are provided. The chimeric viruses are built on the backbone of live attenuated influenza virus (LAIV) which includes deletion of the viral virulence element, the NS1 (non-structural protein 1) (DeLNS1). The chimeric virus strain resulting from DelNS1 live attenuated influenza virus (LAIV), and expressing a CoV2Ag is referred to generally herein, as DelNS1-Sacs-CoV-2-CoV2Ag, with specific chimeric virus differing in name, depe3nding on the CoV-2Ag being expressed. For example, where the chimeric virus expresses RBD from Sars-CoV-2, the chimeric virus is DelNS1-Sars-CoV-2-RBD CoV2Ag. Preferred chimeric vaccine strains include CA04-DelNS1-Sars-CoV-2-RBD; HK68-DelNS1-Sars-CoV-2-RBD; 4801-DelNS1-Sars-CoV-2-RBD and H1N1 (2019)-DelNS1-Sars-CoV-2-RBD strains. A particularly preferred LAIV backbone is a passage adapted strain A/California/04/2009 (a/ca/04/2009; CA04) virus, which includes deletion of the viral virulence element, the NS1 (non-structural protein 1), herein, CA04-DelNS1, and preferably includes two adaptive mutations located in the NP (D101N) and NEP (E95G) genes. The LAIV and DelNS1-Sars-CoV-2-CoV2Ag preferably replicate at low temperatures such as temperatures below 37° C. more preferably between 30 and 33° C. and most preferably, at about 33° C. In some embodiments, the disclosed DelNS1-Sars-CoV-2-CoV2Ag is characterized in that it replicates poorly in MDCK cells at 37° C., when compared to its replication at 33° C. in the MDCK cells. In a particularly preferred embodiment, the mutated DelNS1-Sars-CoV-2-CoV2Ag is able to replicate at levels comparable to wild type influenza virus of the same strain, in a vaccine producing system for example, eggs or MDCK cells. One particularly preferred DelNS1-Sars-CoV-2-CoV2Ag strain is based on CA04-DelNS1, transformed to express receptor binding domain (RBD) of Sars-CoV-2, the chimeric virus referred to herein as CA04-DelNS1-Sars-CoV-2-RBD.

Also disclosed are methods for making chimeric viruses expressing one or more antigens of the Sars-CoV-2. The chimeric virus strains include a LAIV which includes a deletion of the viral virulence element, the NS1 protein and adaptive mutations that allows growth of the mutated strain in vaccine producing systems such as eggs and MDCK cells (i.e., DelNS1-Sars-CoV-2-CoV2Ag strains). The methods include (a) generating a LAIV (which includes a deletion of the coding region of the NS1 coding region), DeLNS1, for example, CA04-DelNS1 (b) expressing an antigen from Sars-CoV-2 (i.e., CoV2Ag) in the DeLNS1, for example, CA04-DelNS1, by transfecting CA04-DelNSI to express the coronavirus antigen in the place of the deleted NS1, hereby generating a chimeric virus, herein DelNS1-Sars-CoV-2-CoV2Ag (b) rescuing DelNS1-Sars-CoV-2-CoV2Ag and (c) passaging rescued virus in one or more vaccine producing cells until viral titer is stabilized, to obtain the DelNS1-Sars-CoV-2-CoV2Ag strain. Exemplary coronavirus antigen domains include receptor binding domain (RBD).

The disclosed methods preferably include reverse genetics. In some preferred embodiments, plasmids containing the deleted NS1 segment (DelNS1) and expressing the selected coronavirus antigen and the other seven genome segments derived from an influenza virus strain, are transfected into 293T/MDCK cell mixture. Rescued virus is passaged in MDCK cells until virus titer is stabilized, with virus titer maintained without meaningful change for three consecutive passages. As used herein, without meaningful change refers to changes including no change or no statistically significant change.

Pharmaceutical compositions are also provided. The pharmaceutical compositions include the disclosed immunogenic DelNS1-Sars-CoV-2-CoV2Ag, such as CA04-DelNS1-CoV2Ag produced according to the disclosed methods. The pharmaceutical compositions typically include an effective amount of a virus to induce an immune response in subject in need thereof when administered to the subject. The pharmaceutical compositions can include additional agents, for example adjuvants to enhance the immune response. In some embodiments, the pharmaceutical compositions do not include an adjuvant. In one embodiment, the composition include an effective mount of the chimeric CA04-DelNS1-CoV2Ag.

Methods of treating a subject in need thereof by administering the pharmaceutical composition to the subject are also provided. The methods can be vaccine protocols. Thus, in some embodiments, the subject is administered the composition to provide prophylactic or therapeutic protection against Sars-CoV-2. The disclosed chimeric CA04-DelNS1-CoV2Ag generated according to the methods disclosed here are administered to a mammal in need thereof by subcutaneous (s.c.), intradermal (i.d.), intramuscular (i.m.), intravenous (i.v.), oral, or intranasal administration; or by injection or by inhalation. In other aspects, the strain is administered intranasally. The compositions containing chimeric DelNS1-CoV2Ag are administrated to a mammal in need of protective immunity against the influenza infection.

### BRIEF DESCRIPTION OF THE DRAWINGS

FIGS. 1A and 1B shows construction of DelNS1-MERS-RBD and DelNS1-MERS-N LAIV.

FIGS. 2A-2C Protection of DPP4 transgenic mice with inoculation of lethal challenge of MERS Coronavirus (2 MLD₅₀).

FIGS. 3A-3C Protection of DPP4 transgenic mice with inoculation of lethal challenge of MERS coronavirus (10 MLD₅₀)

FIGS. 4A and 4B show Sequences of the Receptor Binding Domain (SEQ ID NO:1) of the MERS coronavirus (FIG. 4A) and Receptor Binding Domain (SEQ ID NO:2) Sars-CoV-2 (FIG. 4B).

FIG. 5 shows cloning of Sars-CoV-2 into DelNS1 LAIV vector.

FIG. 6 is a blot showing Verification of NS segment and RBD insert in DelNS1-Sars-CoV-2-RBD vaccine strain

FIG. 7 shows the expression of Sars-CoV-2 RBD in DelNS1-Sars-CoV-2-RBD live attenuated virus infected MDCK cells.

FIG. 8 shows protection of ACE2 transgenic from diseases caused by infection of SARS-CoV-2

### DETAILED DESCRIPTION OF THE INVENTION

### I. Definitions

Materials

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if an aptamer is disclosed and discussed and a number of modifications that can be made to a number of molecules or compositions including the aptamer are discussed, each and every combination and permutation of the aptamer and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials. These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

As used herein, the term “adjuvant” refers to a compound or mixture that enhances an immune response.

As used herein, “attenuated” refers to refers to procedures that weaken an agent of disease (a pathogen). An attenuated virus is a weakened, less vigorous virus. A vaccine against a viral disease can be made from an attenuated, less virulent strain of the virus, a virus capable of stimulating an immune response and creating immunity but not causing illness or less severe illness. Attenuation can be achieved by chemical treatment of the pathogen, through radiation, or by genetic modification, using methods known to those skilled in the art. Attenuation may result in decreased proliferation, attachment to host cells, or decreased production or strength of toxins.

The term “elderly”, as used herein refers to a subject older than 65 years of age.

As used herein, the term “effective amount” or “therapeutically effective amount” means a dosage sufficient to treat, inhibit, or alleviate one or more symptoms of a disease state being treated or to otherwise provide a desired pharmacologic effect. The precise dosage will vary according to a variety of factors such as subject-dependent variables (e.g., age, immune system health, etc.), the disease, and the age of the subject.

As used herein, the term “gene” refers to a nucleic acid (e.g., DNA or RNA) sequence that including coding sequences necessary for the production of a polypeptide, RNA (e.g., including but not limited to, mRNA, tRNA and rRNA) or precursor. The polypeptide, RNA, or precursor can be encoded by a full length coding sequence or by any portion thereof. The term also encompasses the coding region of a structural gene and the sequences located adjacent to the coding region on both the 5′ and 3′ ends for a distance of about 1 kb on either end such that the gene corresponds to the length of the full-length mRNA. The term “gene” encompasses both cDNA and genomic forms of a gene, which may be made of DNA, or RNA. A genomic form or clone of a gene may contain the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

The term “immunogenic composition” or “composition” means that the composition can induce an immune response and is therefore antigenic. By “immune response” means any reaction by the immune system. These reactions include the alteration in the activity of an organism's immune system in response to an antigen and can involve, for example, antibody production, induction of cell-mediated immunity, complement activation, or development of immunological tolerance.

The term “nasal administration” refers to any form of administration whereby an active ingredient is propelled or otherwise introduced into the nasal passages of a subject so that it contacts the respiratory epithelium of the nasal cavity, from which it is absorbed into the systemic circulation. Nasal administration can also involve contacting the olfactory epithelium, which is located at the top of the nasal cavity between the central nasal septum and the lateral wall of each main nasal passage. The region of the nasal cavity immediately surrounding the olfactory epithelium is free of airflow. Thus, specialized methods must typically be employed to achieve significant absorption across the olfactory epithelium.

The terms “oral”, “enteral”, “enterally”, “orally”, “non-parenteral”, “non-parenterally”, and the like, refer to administration of a compound or composition to an individual by a route or mode along the alimentary canal. Examples of “oral” routes of administration of a composition include, without limitation, swallowing liquid or solid forms of a vaccine composition from the mouth, administration of a vaccine composition through a nasojejunal or gastrostomy tube, intraduodenal administration of a vaccine composition, and rectal administration, e.g., using suppositories that release a live bacterial vaccine strain described herein.

The term “mammal” as used herein includes both humans and non-humans and include but is not limited to humans, non-human primates, canines, felines, murines, bovines, equines, and porcines.

The term “topical administration” refers to the application of a pharmaceutical agent to the external surface of the skin or the mucous membranes (including the surface membranes of the nose, lungs and mouth), such that the agent crosses the external surface of the skin or mucous membrane and enters the underlying tissues. Topical administration can result in a limited distribution of the agent to the skin and surrounding tissues or, when the agent is removed from the treatment area by the bloodstream, systemic distribution of the agent. In a preferred form, the agent is delivered by transdermal delivery, e.g., using a transdermal patch. Transdermal delivery refers to the diffusion of an agent across the skin (stratum corneum and epidermis), which acts as a barrier few agents are able to penetrate. In contrast, the dermis is permeable to absorption of many solutes and drugs, and topical administration therefor occurs more readily through skin which is abraded or otherwise stripped of the epidermis to expose the dermis. Absorption through intact skin can be enhanced by combining the active agent with an oily vehicle (e.g., creams, emollients, penetration enhancers, and the like, as described, e.g., in Remington's Pharmaceutical Sciences, current edition, Gennaro et al., eds.) prior to application to the skin (a process known as inunction).

As used herein, the term “peptide” refers to a class of compounds composed of amino acids chemically bound together. In general, the amino acids are chemically bound together via amide linkages (CONH); however, the amino acids may be bound together by other chemical bonds known in the art. For example, the amino acids may be bound by amine linkages. Peptide as used herein includes oligomers of amino acids and small and large peptides, including polypeptides.

As used herein “chimeric virus” refers to a virus stain including viral RNA from more than one type of viral strain.

As used herein, a “variant,” “mutant,” or “mutated” polynucleotide or polypeptide contains at least one polynucleotide or polypeptide sequence alteration as compared to the polynucleotide or polypeptide sequence of the corresponding wild-type or parent polynucleotide or polypeptide. Mutations may be natural, deliberate, or accidental. Mutations include substitutions, deletions, and insertions.

### II. Compositions

Immunogenic compositions including live attenuated chimeric virus are provided, based on DelNS1 live attenuated influenza virus (LAIV) containing a deleted NS1 segment (DelNS1), engineered to express one or more antigens from the novel coronavirus (herein, CoV2Ag). The chimeric Sars-CoV-2 virus can be included in a formulation for administration, in a carrier, and in some embodiments, in combination with an adjuvant. The adjuvant can serve as the carrier. In some embodiments, immunogenic compositions containing the disclosed chimeric virus strains not include an adjuvant. In particularly preferred embodiments, the compositions do not include full length SARS-CoV-2 spike protein or while Sars-CoV-2.

The disclosed chimeric virus strains are based on a DelNS1 live attenuated influenza virus (LAIV) platform which is able to express foreign antigen from the NS1 position of NS segment of the DelNS1 LAIV genome. The compositions are immunogenic in that they can be used to elicit an immune response against the one or more CoV2Ag encoded by the LAIV. The LAIV has improved safety due to deletion of the coding region of the NS1 segment (DelNS1) and adaptive mutations (AM) which improve its growth in vaccine producing systems. Preferred chimeric influenza/CoV2Ag viruses with these combinations of mutations which are based on a passage adapted a/ca/04/2009 (CA04) virus are referred to herein as CA04-DelNS1-CoV2Ag.

A. Live Attenuated Chimeric Virus

The disclosed chimeric viruses can contain various LAIV backbones containing a deleted NS1 segment (DelNS1), engineered to express one or more antigens from the novel coronavirus (herein, CoV2Ag). The resulting chimeric virus resulting from DelNS1 live attenuated influenza virus (LAIV), and expressing a CoV2Ag, is referred to generally herein, as DelNS1-Sars-CoV-2-CoV2AgCoV2Ag.

(i) LAIV Backbones

The backbone virus used to make the disclosed chimeric Sars-CoV-2 are preferably live attenuated influenza A virus strains. Exemplary strains include CA04, and A/WSN/33 and A/PR/8/34. HK4801-DelNS1-SARS-COV-2-RBD AND H1N1 (2019)-DelNS1-SARS-COV-2-RBD exemplified herein can be constructed in the internal gene backbone of CA04-DelNS1 with HA and NA derived from strain of A/HK/4801/2014 (H3N2) or A/HK/2019 (H1N1).

(a) CA04-DelNS1

A preferred LAIV backbone is a mutated influenza virus disclosed in Publication No. 20190125858, incorporated herein by reference. Briefly, cold adapted influenza virus CA04-DelNS1 is based on the 2009 H1N1 influenza stains, and accordingly, includes the that includes a deletion of a virulence factor activity, a first set of one or more mutation(s) that confers replication at 37° C. in the absence of the virulence factor activity, and a second or third set of one or more mutation(s) that confers replication at a temperature below 35° C. The deletion of virulence factor activity can include a deletion of at least part of a virulence factor gene. Such a deletion can be a deletion of at least part of an NS1 gene extending beyond nucleotides 57 to 528 of an NS1 segment of the mutated virus.

The first set of one or more point mutation(s) confer replicative competence, and can lie outside of an M region of the mutated H1N1 influenza virus (for example, a G346A (D101N in protein sequence) mutation in the H1N1 influenza virus genome).

The second set of one or more mutation(s) can include one or more point mutation(s), such as a T261G (L79V in protein sequence) or an A310G (E95G in the protein sequence) mutation in the H1N1 influenza virus genome, positions that have been found to support cold adapted DelNS1 virus replication. The disclosed mutated influenza virus can also include a third set of one or more mutation(s) that confers replication at a temperature below 35° C. These can include one or more point mutation(s) that are distinct from the second set of mutation(s), such as a T261G or an A310G mutation in the H1N1 influenza virus genome. The mutated influenza virus can show reduced replicative ability, relative to a temperature of 35° C. or lower, at a temperature of 37° C. or higher.

(b) A/WSN/33-DelNS1 and A/PR/8/34-DELNS1

The LAIV backbone can be also derived from the A/WSN/33 and A/PR/8/34 strains described in Zheng, et al., J. Virol., 89:10273-10285 (2015). These viral strains include a deletion of the NS1 gene, and an adaptive substitution, A14U (obtained after a few passages of DelNS1 virus), in the 3′ noncoding region (NCR) of the M segment of viral RNA (vRNA) significantly enhances the replication of DelNS1 viruses. The M-A14U substitution supports PR8 DelNS1 virus replication in Vero and MDCK cells, while PR8 DelNS1 virus without this substitution cannot be propagated.

(ii) CoV2Ag

Despite similarities between SARS-CoV and SARS-CoV-2, there is genetic variation between the two and it is not obvious if epitopes that elicit an immune response against SARS-CoV will he effective against SARS-CoV-2.

A preferred CoV2Ag is the receptor binding domain (RBD) of Sars-CoV-2, resulting in the chimeric virus denoted herein as DelNS1-Sars-CoV-2-RBD. The DelNS1-Sars-CoV-2-RBD LAIV platform involves distinguishing features in which the key virulent element, NS1, is knocked out, but DelNS1-Sars-CoV-2-RBD LAIV can still replicate in vaccine production systems (eggs or MDCK cells). When the receptor-binding domain (RBD) of Sar-CoV-2 is inserted into the NS1 site of viral genome, RBD is stably expressed from cells infected with DelNS1-Sars-CoV-2-RBD LAIV.

Use of RBD as antigen minimizes potential antibody-dependent enhancement pathology caused by using full-length spike protein or whole virus as shown in SARS coronavirus. Thus, in preferred embodiments, the antigen is not full length spike protein of Sars-CoV-2. The RBD can be further optimized to cover more than one strain of coronavirus to prevent future emerging coronavirus. DelNS1-Sars-CoV-2-RBD chimeric viruses can induce both neutralizing antibodies and T cell immunities. Various vaccine seeds with different combination of HA and NA of influenza surface proteins can be generated. DelNS1-Sars-CoV-2-RBD chimeric viruses can be produced by engineering an influenza virus with a deleted NS1 segment to express RBD. The resulting chimeric viruses include, but are not limited to CA04-DELNS1-Sars-CoV-2-RBD; HK68-DELNS1-Sacs-CoV-2-RBD; 4801-DELNS1-Sacs-CoV-2-RBD and H1N1 (2019)-DELNS1-Sars-CoV-2-RBD. Therese are all DelNS1-Sars-CoV-2-CoV2AgCoV2Ag, in which the CoV2AgCoV2Ag portion is RBD.

The full genome sequences of CA04-DelNS1-nCoV-RBD were deposited into GenBank and GenBank accession no are MT227009-MT227016. CA04-DelNS1-nCoV-RBD vaccine seed was prepared as disclosed herein was deposited on Apr. 7, 2020 in the American Type Culture Collection (ATCC), 10801 University Boulevard, Manassas, Va. 20110 USA, and given Patent Deposit Number PTA-126682. The disclosed chimeric viruses can be used to prepare a live attenuated vaccine that includes the DelNS1-Sars-CoV-2-CoV2AgCoV2Ag as disclosed under formulations, below.

B. Adjuvants

The disclosed LAIV can be administered in conjunction with other immunoregulatory agents, including adjuvants. Useful adjuvants but are not limited to, one or more set forth below:

Mineral Containing Adjuvant Compositions include mineral salts, such as aluminum salts and calcium salts. Exemplary mineral salts include hydroxides (e.g., oxyhydroxides), phosphates (e.g., hydroxyphosphates, orthophosphates), sulfates, and the like or mixtures of different mineral compounds (e.g., a mixture of a phosphate and a hydroxide adjuvant, optionally with an excess of the phosphate), with the compounds taking any suitable form (e.g., gel, crystalline, amorphous, and the like), and with adsorption to the salt(s) being preferred. The mineral containing compositions can also be formulated as a particle of metal salt (WO/0023105). Aluminum salts can be included in compositions of the invention such that the dose of Al³⁺ is between 0.2 and 1.0 mg per dose.

Oil-Emulsion Adjuvants suitable for use as adjuvants in the invention can include squalene-water emulsions, such as MF59 (5% Squalene, 0.5% Tween 80, and 0.5% Span 85, formulated into submicron particles using a microfluidizer). See, e.g., WO90/14837, Podda, Vaccine 19: 2673-2680, 2001. Additional adjuvants for use in the compositions are submicron oil-in-water emulsions. Examples of submicron oil-in-water emulsions for use herein include squalene/water emulsions optionally containing varying amounts of MTP-PE, such as a submicron oil-in-water emulsion containing 4-5% w/v squalene, 0.25-1.0% w/v Tween 80 (polyoxyethylenesorbitan monooleate), and/or 0.25-1.0% Span 85 (sorbitan trioleate), and, optionally, N-acetylmuramyl-L-alanyl-D-isogluatminyl-L-alanine-2-(1′-2′-dipalmitoyl-s-n-glycero-3-huydroxyphosphophoryloxy)-ethylamine (MTP-PE), for example, the submicron oil-in-water emulsion known as “MF59” (International Publication No. WO90/14837; U.S. Pat. Nos. 6,299,884 and 6,451,325, incorporated herein by reference in their entirety. MF59 can contain 4-5% w/v Squalene (e.g., 4.3%), 0.25-0.5% w/v Tween 80, and 0.5% w/v Span 85 and optionally contains various amounts of MTP-PE, formulated into submicron particles using a microfluidizer such as Model 110Y microfluidizer (Microfluidics, Newton, Mass.). For example, MTP-PE can be present in an amount of about 0-500 μg/dose, or 0-250 μg/dose, or 0-100 μg/dose. Submicron oil-in-water emulsions, methods of making the same and immunostimulating agents, such as muramyl peptides, for use in the compositions, are described in detail in International Publication No. WO90/14837 and U.S. Pat. Nos. 6,299,884 and 6,451,325.

Complete Freund's adjuvant (CFA) and incomplete Freund's adjuvant (IFA) can also be used as adjuvants in the invention.

Saponin Adjuvant Formulations can also be used as adjuvants in the invention. Saponins are a heterologous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations can include purified formulations, such as QS21, as well as lipid formulations, such as Immunostimulating Complexes (ISCOMs; see below).

Saponin compositions have been purified using High Performance Thin Layer Chromatography (HPLC) and Reversed Phase High Performance Liquid Chromatography (RP-HPLC). Specific purified fractions using these techniques have been identified, including QS7, QS17, QS18, QS21, QH-A, QH-B and QH-C. A method of production of QS21 is disclosed in U.S. Pat. No. 5,057,540. Saponin formulations can also comprise a sterol, such as cholesterol (see WO96/33739). Combinations of saponins and cholesterols can be used to form unique particles called ISCOMs. ISCOMs typically also include a phospholipid such as phosphatidylethanolamine or phosphatidylcholine. Any known saponin can be used in ISCOMs. For example, an ISCOM can include one or more of Quil A, QHA and QHC. ISCOMs are described in EP0109942, WO96/11711, and WO96/33739. Optionally, the ISCOMS can be devoid of additional detergent. See WO00/07621. A description of the development of saponin based adjuvants can be found at Barr, et al., “ISCOMs and other saponin based adjuvants”, Advanced Drug Delivery Reviews 32: 247-27, 1998. See also Sjolander, et al., “Uptake and adjuvant activity of orally delivered saponin and ISCOM vaccines”, Advanced Drug Delivery Reviews 32: 321-338, 1998.

Virosomes and Virus-Like Particles (VLPs) can also be used as adjuvants. These structures generally contain one or more proteins from a virus optionally combined or formulated with a phospholipid. They are generally non-pathogenic, non-replicating and generally do not contain any of the native viral genome. The viral proteins can be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, QB-phage (such as coat proteins), GA-phage, fr-phage, AP205 phage, and Ty (such as retrotransposon Ty protein pl).

Bacterial or Microbial Derivatives useful as adjuvants include: (i) Non-Toxic Derivatives of Enterobacterial Lipopolysaccharide (LPS); (ii) lipid derivatives, (iii) immunostimulatory oligonucleotides and ADP-Ribosylating Toxins and Detoxified Derivatives Thereof, (iv) ADP-Ribosylating Toxins and Detoxified Derivatives Thereof. Examples of Non-Toxic Derivatives of LPS Monophosphoryl lipid A (MPL) and 3-O-deacylated MPL (3 dMPL). 3 dMPL is a mixture of 3 De-O-acylated monophosphoryl lipid A with 4, 5 or 6 acylated chains. An example of a “small particle” form of 3 De-O-acylated monophosphoryl lipid A is disclosed in EP 0 689 454. Such “small particles” of 3 dMPL are small enough to be sterile filtered through a 0.22 micron membrane (see EP 0 689 454). Other non-toxic LPS derivatives include monophosphoryl lipid A mimics, such as aminoalkyl glucosaminide phosphate derivatives e.g., RC-529 (Johnson et al., Bioorg Med Chem Lett, 9: 2273-2278, 1999). Examples of lipid A derivatives can include derivatives of lipid A from Escherichia coli such as OM-174. OM-174 is described for example in Meraldi et al., Vaccine 21: 2485-2491, 2003; and Pajak, et al., Vaccine 21: 836-842, 2003. Examples of immunostimulatory oligonucleotides nucleotide sequences containing a CpG motif (a sequence containing an unmethylated cytosine followed by guanosine and linked by a phosphate bond). Bacterial double stranded RNA or oligonucleotides containing palindromic or poly(dG) sequences have also been shown to be immunostimulatory.

The CpG's can include nucleotide modifications/analogs such as phosphorothioate modifications and can be double-stranded or single-stranded. Optionally, the guanosine can be replaced with an analog such as 2′-deoxy-7-deazaguanosine. See Kandimalla, et al., “Divergent synthetic nucleotide motif recognition pattern: design and development of potent immunomodulatory oligodeoxyribonucleotide agents with distinct cytokine induction profiles”, Nucleic Acids Research 31: 2393-2400, 2003; WO02/26757 and WO99/62923 for examples of analog substitutions. The adjuvant effect of CpG oligonucleotides is further discussed in Krieg, Nature Medicine (2003) 9(7): 831-835; McCluskie, et al., FEMS Immunology and Medical Microbiology (2002) 32:179-185; WO98/40100; U.S. Pat. Nos. 6,207,646; 6,239,116 and 6,429,199. The CpG sequence can be directed to Toll-like receptor (TLR9), such as the motif GTCGTT or TTCGTT. See Kandimalla, et al., “Toll-like receptor 9: modulation of recognition and cytokine induction by novel synthetic CpG DNAs”, Biochemical Society Transactions (2003) 31 (part 3): 654-658. The CpG sequence can be specific for inducing a Th1 immune response, such as a CpG-A ODN, or it can be more specific for inducing a B cell response, such a CpG-B ODN. CpG-A and CpG-B ODNs are discussed in Blackwell, et al., J. Immunol. 170: 4061-4068, 2003; Krieg, TRENDS in Immunology 23: 64-65, 2002, and WO01/95935. In some aspects, the CpG oligonucleotide can be constructed so that the 5′ end is accessible for receptor recognition. Optionally, two CpG oligonucleotide sequences can be attached at their 3′ ends to form “immunomers”. See, for example, Kandimalla, et al., BBRC 306: 948-95, 2003; Kandimalla, et al., Biochemical Society Transactions 31: 664-658, 2003; Bhagat et al., “BBRC 300: 853-861, 2003, and WO03/035836. Bacterial ADP-ribosylating toxins and detoxified derivatives thereof can be used as adjuvants in the invention. For example, the toxin can be derived from E. coli (i.e., E. coli heat labile enterotoxin (LT)), cholera (CT), or pertussis (PTX). The use of detoxified ADP-ribosylating toxins as mucosal adjuvants is described in WO95/17211 and as parenteral adjuvants in WO98/42375. In some aspects, the adjuvant can be a detoxified LT mutant such as LT-K63, LT-R72, and LTR192G. The use of ADP-ribosylating toxins and detoxified derivatives thereof, particularly LT-K63 and LT-R72, as adjuvants can be found in the following references, each of which is specifically incorporated by reference herein in their entirety: Beignon, et al., Infection and Immunity 70: 3012-3019, 2002; Pizza, et al., Vaccine 19: 2534-2541, 2001; Pizza, et al., Int. J. Med. Microbiol 290: 455-461, 2003; Scharton-Kersten et al., Infection and Immunity 68: 5306-5313, 2000; Ryan et al., Infection and Immunity 67: 6270-6280, 2003; Partidos et al., Immunol. Lett. 67: 09-216, 1999; Peppoloni et al., Vaccines 2: 285-293, 2003; and Pine et al., J. Control Release 85: 263-270, 2002.

Bioadhesives and mucoadhesives can also be used as adjuvants in the invention. Suitable bioadhesives can include esterified hyaluronic acid microspheres (Singh et al., J. Cont. Rel. 70:267-276, 2001) or mucoadhesives such as cross-linked derivatives of poly(acrylic acid), polyvinyl alcohol, polyvinyl pyrollidone, polysaccharides and carboxymethylcellulose. Chitosan and derivatives thereof can also be used as adjuvants in the invention disclosed for example in WO99/27960.

Adjuvant Microparticles: Microparticles can also be used as adjuvants. Microparticles (i.e., a particle of about 100 nm to about 150 μm in diameter, or 200 nm to about 30 μm in diameter, or about 500 nm to about 10 μm in diameter) formed from materials that are biodegradable and/or non-toxic (e.g., a poly(alpha-hydroxy acid), a polyhydroxybutyric acid, a polyorthoester, a polyanhydride, a polycaprolactone, and the like), with poly(lactide-co-glycolide) are envisioned, optionally treated to have a negatively-charged surface (e.g., with SDS) or a positively-charged surface (e.g., with a cationic detergent, such as CTAB).

Examples of liposome formulations suitable for use as adjuvants are described in U.S. Pat. Nos. 6,090,406, 5,916,588, and EP 0 626 169.

Additional adjuvants include polyoxyethylene ethers and polyoxyethylene esters. WO99/52549. Such formulations can further include polyoxyethylene sorbitan ester surfactants in combination with an octoxynol (WO 01/21207) as well as polyoxyethylene alkyl ethers or ester surfactants in combination with at least one additional non-ionic surfactant such as an octoxynol (WO 01/21152). In some aspects, polyoxyethylene ethers can include: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, or polyoxyethylene-23-lauryl ether.

PCPP formulations for use as adjuvants are described, for example, in Andrianov et al., Biomaterials 19: 109-115, 1998.1998. Examples of muramyl peptides suitable for use as adjuvants in the invention can include N-acetyl-muramyl-L-threonyl-D-isoglutamine (thr-MDP), N-acetyl-normuramyl-1-alanyl-d-isoglutamine (nor-MDP), and N-acetylmuramyl-1-alanyl-d-isoglutaminyl-1-alanine-2-(1′-2′-dipalmitoyl-s-n-glycero-3-hydroxyphosphoryloxy)-ethylamine MTP-PE). Examples of imidazoquinolone compounds suitable for use as adjuvants in the invention can include Imiquimod and its homologues, described further in Stanley, “Imiquimod and the imidazoquinolones: mechanism of action and therapeutic potential” Clin Exp Dermatol 27: 571-577, 2002 and Jones, “Resiquimod 3M”, Curr Opin Investig Drugs 4: 214-218, 2003. Human immunomodulators suitable for use as adjuvants in the invention can include cytokines, such as interleukins (e.g., IL-1, IL-2, IL-4, IL-5, IL-6, IL-7, IL-12, and the like), interferons (e.g., interferon-gamma), macrophage colony stimulating factor, and tumor necrosis factor.

Adjuvant Combinations: The adjuvants are used in come preferred embodiments as combinations. For example, adjuvant compositions can include: a saponin and an oil-in-water emulsion (WO99/11241); a saponin (e.g., QS21)+a non-toxic LPS derivative (e.g., 3 dMPL) (see WO94/00153); a saponin (e.g., QS21)+a non-toxic LPS derivative (e.g., 3 dMPL)+a cholesterol; a saponin (e.g., QS21)+3 dMPL+IL-12 (optionally+a sterol) (WO98/57659); combinations of 3 dMPL with, for example, QS21 and/or oil-in-water emulsions (See European patent applications 0835318, 0735898 and 0761231); SAF, containing 10% Squalane, 0.4% Tween 80, 5% pluronic-block polymer L121, and thr-MDP, either microfluidized into a submicron emulsion or vortexed to generate a larger particle size emulsion. Ribi adjuvant system (RAS), (Ribi Immunochem) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox); and one or more mineral salts (such as an aluminum salt)+a non-toxic derivative of LPS (such as 3 dPML).

Aluminum salts and MF59 are examples of adjuvants for use with injectable influenza vaccines. Bacterial toxins and bioadhesives are examples of adjuvants for use with mucosally-delivered vaccines, such as nasal vaccines. All adjuvants noted above and others as generally known in the art to one of ordinary skill can be formulated for intranasal administration using techniques well known in the art.

C. Formulations and Carriers

The composition of the invention can be formulated in pharmaceutical compositions. These compositions can comprise, in addition to one or more of the DelNS1-Sars-CoV-2-CoV2AgCoV2Ag, a pharmaceutically acceptable excipient, carrier, buffer, stabilizer, or other materials well known to those skilled in the art. Such materials should typically be non-toxic and should not typically interfere with the efficacy of the active ingredient. The precise nature of the carrier or other material can depend on the route of administration, e.g., oral, intravenous, cutaneous or subcutaneous, nasal, intramuscular, or intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil, or synthetic oil. Physiological saline solution, dextrose, or other saccharide solution or glycols such as ethylene glycol, propylene glycol, or polyethylene glycol can be included. The term “carrier” refers to a diluent, adjuvant, excipient, or vehicle with which the pharmaceutical composition (e.g., immunogenic or vaccine formulation) is administered. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, ethanol and the like. Examples of suitable pharmaceutical carriers are described in “Remington's Pharmaceutical Sciences” by E. W. Martin. The formulation should be selected according to the mode of administration.

For intravenous, cutaneous, or subcutaneous injection, or injection at the site of affliction, the active ingredient will be in the form of a parenterally acceptable aqueous solution which is pyrogen-free and has suitable pH, isotonicity, and stability. Those of relevant skill in the art are well able to prepare suitable solutions using, for example, isotonic vehicles such as Sodium Chloride Injection, Ringer's Injection, or Lactated Ringer's Injection. Preservatives, stabilizers, buffers, antioxidants, and/or other additives can be included, as required.

Administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of disease being treated. Prescription of treatment, e.g., decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in the latest edition of Remington's Pharmaceutical Science, Mack Publishing Company, Easton, Pa. (“Remington's”).

### III. Methods of Making

A protocol to engineer DelNS1-Sars-CoV-2-CoV2Ag chimeric virus is provided for in the examples section of Published Application No. 20190125858, incorporated herein by reference. The protocol includes (a) generating an influenza virus for example the California(CA)/04/09 strain with the coding region of the NS1 gene removed from its genome. The coding region of the NS1 gene can be removed using methods known in the art. Methods to introduce targeted mutations into a genome or, in the context of virology, into a virus are subsumed under the term reverse genetics (RG) and are disclosed for example, in Hoffmann et al., Proc Natl Acad Sci USA, 97(11):6108-13 Zheng et al., J. Virol. 89(20): 10273-85 and Dauber, et al., J. Virol., 78(4):1865-1872 (2004), the materials and method of which are incorporated herein by reference. The method of generating influenza virus with the NS1 coding region deleted, as disclosed in Published Application No. 20190125858 is generalized and summarized herein.

A. Generating Live Attenuated Influenza Virus (LAIV) with the Coding Region of the NS1 Gene Removed

The LAIV can be constructed as disclosed herein in the Examples, with the methods disclosed herein exemplifying CA04-DelNS1. Briefly, an NS1 deletion Plasmid is constructed. Construction of NS1 Deletion Plasmid: A suitable viral strain, for example, 2009 H1N1 A/California/04/09 (CA04) can be used as backbone to construct the DelNS1 vaccines strain. Plasmid without NS1 expression can be constructed by inverse PCR with primers as follows:

CA04-DelNS1-56F:
(SEQ ID NO: 3)
GACATACTTATGAGGATGTC;
CA04-DelNS1-529F:
(SEQ ID NO: 4)
CTGAAAGCTTGACATGGTGTTG.

These primers can be used to construct CA4-DelNS1 virus from a California(CA)/04/09 strain through reverse genetic procedures that deleted an intron at 56-529.

Primers
(SEQ ID NO: 5)
5′-GACATACTGTGAGGATGTCAAAAATG-3- =
(NS-529F)
and
(SEQ ID NO: 6)
5 = -CTGAAAGCTTGACACAGTGTTTGG-3′;
(NS-56R)

can be used to construct A/WSN/33-DelNS1 and A/PR/8/34-DELNS1.

The NS1 deletion plasmid can be constructed according to the protocol described in a previous report (Garcia-Sastre, J. Virology 252:324-330, 1998); Zheng, et al., J Virol 89:10273-10285 (2015). In brief, inverse PCR is carried out to delete the intron of the NS gene inserted into the pHW2000 vector and the plasmid phosphorylated and self-ligated. For point mutations, commercially available kits can be used, for example, the QuikChange II site-directed mutagenesis kit (Stratagene).

(i) Rescue of CA-04-DELNS1 Virus

Nine plasmids: pHW2000-CA04-PB2, pHW2000-CA04-PB1, pHW2000-CA04-PA, pHW2000-CA04-NP, pHW2000-CA04-HA, pHW2000-CA04-NA, pHW2000-CA04-M, pHW2000-CA04-DelNS1 and pCX-CA04-NS1 are mixed together in one tube. Each one is present at 1 Transfection with the mixed plasmids was conducted in 80% confluent 293T cells plated in a 6-well plate. During transfection the old medium was replaced with 1 ml Opti-MEM without penicillin and streptomycin. Sixteen hours later the supernatant was discarded and 2 ml of MEM containing 1 μg/ml trypsin was added. Seventy hours after transfection, the supernatant was collected after the cell debris was removed.

(ii) Passage of DelNS1 Virus

Two hundred microliter rescued DelNS1 virus can be injected into a 9 to 10-day-old fertilized egg and incubated in the 37° C. incubator for 48 hours. Egg allantoic fluid was collected and HA titer was measured. Blood cells and other debris were removed by centrifugation at 1500 g for 10 minutes. Supernatant was transferred into a Millipore 100K ultra filter and centrifuged at the speed of 3000 g for 10 minutes. PBS was added to the filter to give a volume of 10 ml to wash the concentrated virus, and the suspension was again centrifuged at 3000 g for 10 minutes. Two hundred microliter of the resulting virus preparation is used to inoculate 9 to 10-day-old fertilized eggs and the procedure was repeated until the virus HA titer increased dramatically.

Rescued DelNS1-Sars-CoV-2-CoV2Ag chimeric virus can be cultured in any virus-producing cell until virus titer is stabilized, evidenced for example, when the virus titer remains unchanged for at least three consecutive passages in MDCK cells and eggs. Supernatant from the transfected cells after 72 hours is collected and passaged in MDCK cells.

A preferred cell for passaging is MDCK (Madin-Darby canine kidney) cells. However, the cells used for the cultivation of viruses using a cultivation medium can be cells that can grow in vitro in synthetic media and can be used for the propagation of viruses. These can be for example BSC-1 cells, LLC-MK cells, CV-1 cells, CHO cells, COS cells, murine cells, human cells, HeLa cells, 293 cells, VERO cells, MDBK cells, MDOK cells, CRFK cells, RAF cells, TCMK cells, LLC-PK cells, PK15 cells, WI-38 cells, MRC-5 cells, T-FLY cells, BHK cells, SP2/0 cells, NS0, PerC6 (human retina cells), chicken embryo cells or derivatives, embryonated egg cells, embryonated chicken eggs or derivatives thereof.

The cultivation medium used for the production of viruses can be any medium known from prior art that is applicable for virus cultivation. Preferably the medium is a synthetic medium. This can be for example basal media as Modified Eagle's media MEM, minimum essential media MEM, Dulbecco's modified Eagle's media D-MEM, D-MEM-F12 media, William's E media, RPMI media and analogues and derivative thereof. These can also be specialty cell cultivation and virus growth media as VP-SFM, OptiPro TM SFM, AIM V® media, HyQ SFM4 MegaVir™, EX-CELL™ Vero SFM, EPISERF, ProVero, any 293 or CHO media and analogues and derivatives thereof. These media can be supplemented by any additive known from prior art that is applicable for cell and virus cultivation as for example animal sera and fractions or analogues thereof, amino acids, growth factors, hormones, buffers, trace elements, trypsin, sodium pyruvate, vitamins, L-glutamine and biological buffers. Preferable medium is OptiPRO™ SFM supplemented with L-glutamine and trypsin.

Thus, disclosed method includes culturing the virus in for an effective amount of time to obtain a stable viral titer. In preferred embodiment, the rescued virus is passaged in a virus-producing cell, for example, MDCK cells for a period of time until viral titre remains unchanged for 3 consecutive passaged. This culture period can range from 10-50 passages, preferably, for over 20 passages at 33° C. The time and conditions of culture result in adaptive mutations, which allows replication of the LAIVB in vaccine producing systems such as eggs or MDCK. The examples DelNS1-Sars-CoV-2-RBD can replicate in the vaccine producing cell line, MDCK cells, for the viral strain tested.

(iii) Construction of the Sars-CoV-2-CoV2Ag Plasmid

A plasmid including Sars-CoV-2 antigen can be prepared as exemplified here for Sars-CoV-2-RBD.

Construction of the pHW2000-Sars-CoV-2-RBD-NEP Plasmid

The plasmid including the Sars-CoV-2-RBD can be prepared adapting the method disclosed for the pHW2000-MERS-RBD-NEP Plasmid in U.S. Published application No. 2019/0125858.

Briefly, to generate recombinant NS1-deleted influenza virus expressing Sars-CoV-2 receptor binding domain (RBD), a pHW2000-Sars-CoV-2-RBD-NEP plasmid can be constructed. It has an open reading frame which is composed of CA04 N terminal of NS1, Sars-CoV-2 RBD domain, PTV1-2A cleavage site, CA04 NEP with the mutated N terminal NS1 sequence.

The sequence of Sars-CoV-2-RBD-PTV1-2A is amplified by PCR and inserted into the pHW2000-CA04-DelNS1, which contains only CA04 NEP open reading frame, by ligation independent cloning using exonuclease III. After transformation, plasmids were extracted from right clones and subsequently sequenced to confirm the sequence.

(iv) Rescue of the DELNS1-Sars-CoV-2CoV2Ag Chimeric Virus

Rescue of the DELNS1-Sars-CoV-2CoV2Ag chimeric virus is exemplified herein using the CA04-delNS1-RBD Virus. These methods are applicable to rescue of chimeric virus using other LAIV backbone, for example, HK68-DELNS1-Sars-CoV-2-RBD; 4801-DELNS1-Sars-CoV-2-RBD and H1N1 (2019)-DELNS1-Sars-CoV-2-RBD.

Rescue of the CA04-delNS1-RBD Virus

Nine plasmids: pHW2000-CA04-PB2, pHW2000-CA04-PB1, pHW2000-CA04-PA, pHW2000-CA04-NP, pHW2000-CA04-HA, pHW2000-CA04-NA, pHW2000-CA04-M, pHW2000-Sars-CoV-2-RBD-NEP and pCX-CA04-NS1, each with 1 μg, are mixed and used to transfect 80% confluent 293T cells in a 6-well plate. During transfection the old medium is replaced with 1 ml of Opti-MEM without antibiotics. Sixteen hours later the supernatant is discarded and 2 ml of MEM containing 1 μg/ml trypsin added. Seventy hours after transfection, the supernatant was collected after the cell debris is removed. The supernatant is injected into 9 to 10-day-old fertilized eggs and incubated at 37° C. for 48 hours. Egg allantoic fluid is collected, and cleared by centrifugation. The virus is then sequenced and titered by plaque assay in MDCK cells.

### IV. Methods of Use

The disclosed DelNS1-Sars-CoV-2-CoV2Ag chimeric virus can be used to effectively increase viral titer or elicit an immune response in a subject in need thereof. In some aspects, subjects can include the elderly (e.g., >65 years old), young children (e.g., <5 years old). Methods for improving immune response in children using adjuvanted formulations are disclosed for example in U.S. Publication 2017/0202955.

The DelNS1-Sars-CoV-2-CoV2Ag chimeric virus stains can generally be administered directly to a mammal in need thereof to increase viral titer in the mammal and elicit an immune response. In some embodiments the subject is a young child, less than 5 years of age. In other embodiments, the subject is a young child, less than two years of age. In the embodiments, the composition is administered intranasally. In other embodiments the subject is elderly, and the subject can be between the ages of 5 and 65.

Viruses are typically administered to a patient in need thereof in a pharmaceutical composition. Pharmaceutical compositions containing virus may be for systemic or local administration. Dosage forms for administration by parenteral (intramuscular (IM), intraperitoneal (IP), intravenous (IV) or subcutaneous injection (SC)), or transmucosal (nasal, vaginal, pulmonary, or rectal) routes of administration can be formulated. In the most preferred embodiments, the immunizing virus is delivered peripherally by intranasally or by intramuscular injection, and the therapeutic virus is delivered by local injection.

Direct delivery can be accomplished by parenteral injection (e.g., subcutaneously, intraperitoneally, intradermal, intravenously, intramuscularly, or to the interstitial space of a tissue), or mucosally, such as by rectal, oral (e.g., tablet, spray), vaginal, topical, transdermal (See e.g., WO99/27961) or transcutaneous (See e.g., WO02/074244 and WO02/064162), inhalation, intranasal (See e.g., WO03/028760), ocular, aural, pulmonary or other mucosal administration. Compositions can also be administered topically by direct transfer to the surface of the skin. Topical administration can be accomplished without utilizing any devices, or by contacting naked skin with the composition utilizing a bandage or a bandage-like device (see, e.g., U.S. Pat. No. 6,348,450). In some aspects, the mode of administration is parenteral, mucosal, or a combination of mucosal and parenteral immunizations. In other aspects, the mode of administration is parenteral, mucosal, or a combination of mucosal and parenteral immunizations in a total of 1-2 vaccinations 1-3 weeks apart. In related aspects, the route of administration includes but is not limited to intranasal delivery.

A. Effective Amounts

Typically the composition is administered in an effective amount to induce an immune response against a one or more Sars-CoV-2 antigens encoded by the chimeric virus. For example, an effective amount of virus generally results in production of antibody and/or activated T cells that kill or limit proliferation of or infection by the Sars-CoV-2.

The composition can typically be used to elicit systemic and/or mucosal immunity, for example to elicit an enhanced systemic and/or mucosal immunity. For example, the immune response can be characterized by the induction of a serum IgG and/or intestinal IgA immune response. Typically, the level of protection against influenza infection can be more than 50%, e.g., 60%, 70%, 80%, 90%, 91%, 92%, 93%, 94%, 95%, 96%, 97%, 98%, 99%, or more. In one aspect, the level of protection can be 100%.

The immune response induced by the invention can be one or both of a TH1 immune response and a TH2 response. The immune response can be an improved or an enhanced or an altered immune response. The immune response can be one or both of a systemic and a mucosal immune response. For example, the immune response can be an enhanced systemic and/or mucosal response. An enhanced systemic and/or mucosal immunity is reflected in an enhanced TH1 and/or TH2 immune response. For example, the enhanced immune response can include an increase in the production of IgG1 and/or IgG2a and/or IgA. In another aspect the mucosal immune response can be a TH2 immune response. For example, the mucosal immune response can include an increase in the production of IgA.

Typically, activated TH2 cells enhance antibody production and are therefore of value in responding to extracellular infections. Activated TH2 cells can typically secrete one or more of IL-4, IL-5, IL-6, and IL-10. A TH2 immune response can also result in the production of IgG1, IgE, IgA, and/or memory B cells for future protection. In general, a TH2 immune response can include one or more of an increase in one or more of the cytokines associated with a TH2 immune response (such as IL-4, IL-5, IL-6 and IL-10), or an increase in the production of IgG1, IgE, IgA and memory B cells. For example, an enhanced TH2 immune response can include an increase in IgG1 production. A TH1 immune response can include one or more of an increase in CTLs, an increase in one or more of the cytokines associated with a TH1 immune response (such as IL-2, IFN-gamma, and TNF-alpha), an increase in activated macrophages, an increase in NK activity, or an increase in the production of IgG2a. For example, the enhanced TH1 immune response can include an increase in IgG2a production.

The DelNS1-Sars-CoV-2-CoV2Ag chimeric virus strains can be used either alone or in combination with other agents optionally with an immunoregulatory agent capable of eliciting a Th1 and/or Th2 response.

B. Dosages

The precise dosage will vary according to a variety of factors such as subject-dependent variables (e.g., age, immune system health, etc.), and age of the subject being treated. Appropriate dosages can be determined by a person skilled in the art, considering the therapeutic context, age, and general health of the recipient. The selected dosage depends upon the desired therapeutic effect, on the route of administration, and on the duration of the treatment desired. In determining the effective amount of the virus to be administered for the prophylaxis, the physician may evaluate circulating plasma levels of virus, and/or the production of existing antibodies against the antigen(s). Active virus can also be measured in terms of plaque-forming units (PFU). A plaque-forming unit can be defined as areas of cell lysis (CPE) in monolayer cell culture, under overlay conditions, initiated by infection with a single virus particle. Generally, dosage levels of virus between 10² and 10¹² pfu are administered to humans. In different embodiments, the dosage range is from 10⁴ to 10¹⁰ pfu, 10⁵ to 10⁹ pfu, 10⁶ to 10⁸ pfu, or any dose within these stated ranges. When more than one vaccine is to be administered (i.e., in combination vaccines), the amount of each vaccine agent can be within their described ranges.

Virus is typically administered in a liquid suspension, in a volume ranging between 10 μl and 100 μl depending on the route of administration. Vaccine volumes commonly practiced range from 0.1 mL to 0.5 mL. Generally, dosage and volume will be lower for local injection as compared to systemic administration or infusion.

The vaccine composition can be administered in a single dose or a multi-dose format. Vaccines can be prepared with adjuvant hours or days prior to administrations, subject to identification of stabilizing buffer(s) and suitable adjuvant composition. Typically, the dose will be 100 μl administered locally in multiple doses, while systemic or regional administration via subcutaneous, intramuscular, intra-organ, intravenous or intranasal administration can be from for example, 10 to 100 μl.

### V. Kits

A kit including the disclosed DelNS1-Sars-CoV-2-CoV2Ag chimeric virus strains are also provided. The kit can include a separate container containing a suitable carrier, diluent or excipient. Additionally, the kit can include instructions for mixing or combining ingredients and/or administration.

Compositions can be in liquid form or can be lyophilized. Suitable containers for the compositions include, for example, bottles, vials, syringes, and test tubes. Containers can be formed from a variety of materials, including glass or plastic. A container can have a sterile access port (for example, the container can be an intravenous solution bag or a vial having a stopper pierceable by a hypodermic injection needle).

The kit can further include a second container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery device(s). The kit can further include a third component comprising an adjuvant.

The kit can also include a package insert containing written instructions for methods of inducing immunity, preventing infections, or for treating infections. The package insert can be an unapproved draft package insert or can be a package insert approved by the Food and Drug Administration (FDA) or other regulatory body. The invention also provides a delivery device pre-filled with the compositions of the invention.

The compositions are generally formulated as sterile, substantially isotonic and in full compliance with all Good Manufacturing Practice (GMP) regulations of the U.S. Food and Drug Administration.

The disclosed compositions, and methods can be further understood through the following numbered paragraphs

1. A live attenuated chimeric virus comprising (a) an influenza virus genome, wherein the influenza virus genome comprises a deletion of a virulence factor activity, and optionally, a first set of one or more mutation(s) that confers replication at 37° C. in the absence of the virulence factor activity; and a second set of one or more mutation(s) that confers replication at a temperature below 35° C., and (b) an insertion of one or more genes encoding one or more Sars-CoV-2 antigens (CoV2Ag).

2. The attenuated chimeric virus of paragraph 1, wherein the influenza virus genome is from an influenza virus A subtype H1N1 or H3N2.

3. The attenuated chimeric virus of paragraph 2, wherein the influenza virus genome is from an influenza virus A subtype H1N1 or H3N2 strain selected from the group consisting of CA04 (A/California/04/2009); HK68 (strain A/Hong Kong/1/68), 4801 (H3N2 A/HK/4801/2014), H1N1 (2019); A/WSN/33 and A/PR/8/34.

4. The attenuated chimeric virus any one of paragraphs 1-3, wherein the deletion of virulence factor activity comprises a deletion of at least part of a virulence factor gene.

5. The chimeric virus of any one of paragraphs 1-4, wherein the deletion comprises a deletion of at least part of Non-Structural Protein 1 (NS1) gene extending beyond nucleotides 57 to 528 of an NS1 segment of the mutated virus.

6. The chimeric virus of any one of paragraphs 1-5, comprising a first set of one or more mutation(s), wherein the first set of one or more mutation(s) comprises a first set of one or more point mutation(s) that confer replicative competence.

7. The chimeric virus any one of paragraphs 1-5, wherein the first set of one or more point mutation(s) lies outside of an M region of the mutated influenza virus.

8. The chimeric virus of paragraph 3, wherein the influenza virus genome is from the A/California/04/2009 influenza strain, and at least one of the first set of one or more point mutation(s) is a G346A mutation in the viral genome.

9. The chimeric virus of any one of paragraphs 1-8, wherein the virus replicates poorly in MDCK cells at 37° C., when compared to its replication at 33° C. in the MDCK cells.

10. The chimeric virus of any one of paragraphs 1-7, wherein the second set of one or more mutation(s) comprises a second set of one or more point mutation(s).

11. The chimeric virus of paragraph 3, wherein the first set of one or more mutation(s) comprises an A14U substitution in the 3′ noncoding region of the M segment of viral RNA.

12. The chimeric virus of any one of paragraphs 1-9, wherein at least one member of the second set of one or more point mutation(s) is selected from the group consisting of a T261G and an A310G mutation in the influenza virus genome.

13. The chimeric virus of paragraph 12, comprising a third set of one or more mutation(s) that confers replication at a temperature below 35° C.

14. The chimeric virus of paragraph 12, wherein the third set of one or more mutation(s) comprises a third set of one or more point mutation(s) that is distinct from the second set of one or more point mutation(s), and is selected from the group consisting of a T261G and an A310G mutation in the H1N1 influenza virus genome.

15. The chimeric virus of any one of paragraphs 1-13, wherein the one or more CoV2Ag is the Sar-CoV-2 receptor binding domain (RBD).

16. The chimeric virus of any one of paragraph 1-15, selected from the group consisting of CA04-DelNS1-Sars-CoV-2-RBD; HK68-DelNS1-Sars-CoV-2-RBD; 4801-DelNS1-Sars-CoV-2-RBD and H1N1 (2019)-DelNS1-Sacs-CoV-2-RBD.

17. A pharmaceutical composition comprising an effective amount of the chimeric virus of anyone of paragraphs 1-16.

18. The composition of paragraph 17, further comprising an adjuvant.

19. The composition of any one of paragraphs 17 or 18, suitable for nasal administration.

20. A method for increasing an immune response to Sars-CoV-2 in a subject in need thereof, comprising administering the composition of any one of paragraphs 1-13, to the subject.

### EXAMPLES

### Materials and Methods

### Cells and Viruses—

All cell lines were obtained from ATCC. Human cells were maintained in Dulbecco's minimal essential medium (DMEM) supplemented with 10% fetal bovine serum, 100 units/ml penicillin, and 100 μg/ml streptomycin sulfate (Life Technologies). MDCK cells were cultured in Eagle's minimal essential medium (MEM) supplemented with the same amount of serum and antibiotics. CA04-DelNS1 LAIV were constructed and rescued according to the protocols described here and in the previous report (Wang, et al, mBio, 10(5): e02180-19 (2019). Viral gene segments were amplified and cloned into pHW2000 plasmids, resulting in eight pHW2000 plasmids, which were transfected into 293T/MDCK cell mixtures. Rescued virus was amplified in MDCK cells or embryonated chicken eggs. CA04-DelNS1 LAIV was used as backbone for making other DelNS1-SARS-CoV-RBD LAIVs.

Construction of Plasmids

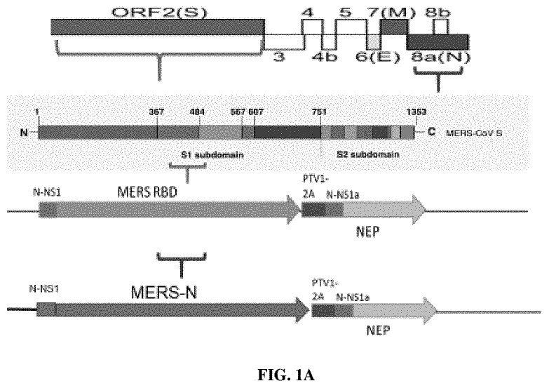

Plasma construction follows the protocol described in Wang, et al, mBio, 10(5): e02180-19 (2019). NS1 deletion plasmid pHW2000-DelNS1 was constructed as described before (Zheng, et al, J. Virol., 89:10273-10285 (2015)). Inverse PCR is performed to delete the NS1 gene using plasmid pHW2000-CA04-NS (influenza A virus). The PCR product was then gel purified, phosphorylated and self-ligated using a standard protocol. Primers for CA04-DeNS1 inverse PCR are 5′-GACATACTTATGAGGATGTC-3′ (SEQ ID NO:3 (CA04-DelNS1-F) and 5′-CTGAAAGCTTGACATGGTGTTG-3′ (SEQ ID NO:4) (CA04-DelNS1-R) (Wang, et al, mBio, 10(5): e02180-19 (2019). A QuikChange II site-directed mutagenesis kit (Agilent) is used to generate point mutations. pHW2000-CA4-DelNS1-SARS-CoV2-RBD was made by cloning of the RBD region of SARS-CoV-2 into the site of deleted NS1 of CA04-DelNS1. A protease cleavage motif, 2A, was inserted between RBD and the NEP coding region (FIG. 1).

HK68-DelNS1-SARS-CoV-2-RBD was constructed using the backbone of CA04-DelNS1 with hemagglutinin (HA) and neuraminidase (NA) derived from A/HK/01/1968 (H3N2). Similarly, HK4801-DelNS1-SARS-COV-2-RBD AND H1N1 (2019)-DelNS1-SARS-COV-2-RBD were constructed in the internal gene backbone of CA04-DelNS1 with HA and NA derived from strain of A/HK/4801/2014 (H3N2) or A/HK/2019 (H1N1).

Generation and Passage of DelNS1 Viruses.

Eight pHW2000 plasmids containing the DelNS1 segment and the other 7 influenza virus genomic segments, together with an NS1 expression plasmid, were transfected into a 293T/MDCK cell mixture and incubated overnight. The DNA mixture was removed and MEM supplemented with 1 μg/m1N-tosyl-L-phenylalanine chloromethyl ketone (TPCK)-treated trypsin (Sigma) added. Virus supernatant was collected 72 h later and designated passage 0 (P0) virus and subsequently passaged in MDCK cells or embryonated chicken eggs. For CA04-DelNS1 virus, rescued virus was passaged in MDCK cells 10 times at 37° C. and then a further 10 times at 30° C. CA04-DelNS1-SARS-CoV-2-RBD, HK68-DelNS1-SARS-CoV-2-RBD, HK4801-DelNS1-SARS-COV-2-RBD AND H1N1 (2019)-DelNS1-SARS-COV-2-RBD were rescued and passage similarly as described above.

For all DelNS1-SARS-CoV-2 RNA LAIV viruses, insertion of RBD and deletion of the NS1 gene was confirmed by reverse transcription-PCR (RT-PCR) and sequencing.

RT-PCR

Verification of NS Segment and RBD Insert in DelNS1-nCoV-RBD Vaccine Strain

RNA was extracted from DelNS1 vaccine strains, (CA04-DelNS-Sars-CoV-2-RBD, (herein also, CA04-DelNS-nCoV-RBD), HK68-DelNS1-Sars-CoV-2-RBD (herein, also, HK68-DelNS1-nCoV-RBD), 4801-DelNS1-Sars-CoV-2-RBD (herein, also, 4801-DelNS1-nCoV-RBD) and H1N1 (2019)-DelNS1-Sars-CoV-2-RBD (herein, also, H1N1 (2019)-DelNS1-nCoV-RBD), and subsequently, passaged in eggs. RT-PCR with primers specific for the NS segment and RBD of Sars-CoV-2 (herein also, nCoV) were performed and PCR products were analyzed be agarose electrophoresis. Correct size of PCR products, NS and RBD were observed from all DelNS1 vaccine strains.

Verification of expression of Sars-CoV-2 (nCoV) RBD in DelNS1-nCoV-RBD live attenuated virus infected MDCK cells. MDCK Cells were infected with CA04-DelNS-nCoV-RBD, HK68-DelNS1-nCoV-RBD, 4801-DelNS1-nCoV-RBD, or H1N1 (2019)-DelNS1-nCoV-RBD at 0.1 MOI, or mock infection for 16 hours. Cell lysates were harvested and analyzed by Western blot using either anti-NP (for viral protein NP) or anti-V5 (for RBD which is tagged with a V5 epitope). As shown in the results, RBD was expressed from all DelNS1 vaccines strains.

Animal Studies

Two groups (six each) of six to eight-week old female DPP4 transgenic mice are anesthetized and then inoculated intranasally with 25 μl PBS containing 5×10⁵ TCID₅₀ of MERS-RBD-DelNS1, DelNS1-MERS-N or control (PBS only), twice, respectively, four weeks apart. Mice were challenged with MERS coronavirus (500 pfu=10 MLD₅₀ or 100 pfu=2 MLD₅₀). Mice were monitored for 14 days for body weight loss and mortality.

### Example 1. Construction of DelNS1-MERS-RBD and DelNS1-MERS-N LAIV Vaccine Strains

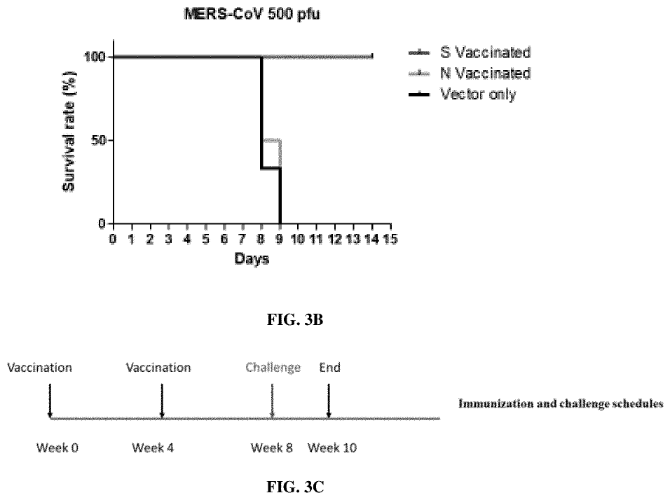

For proof of concept, gene segments containing the RBD and N derived from MERS coronavirus was cloned into NS segment of CA04-DelNS1 LAIV (Wang et al., mBio 10 (5):e12180-19 (2019).) (FIGS. 1A-1B). The sequences for the Receptor Binding Domain (RBD) of the MERS coronavirus is shown in FIG. 4A.

### Example 2. Protection of DPP4 Transgenic Mice with Inoculation of Lethal Challenge of MERS Coronavirus (2 MLD₅₀)

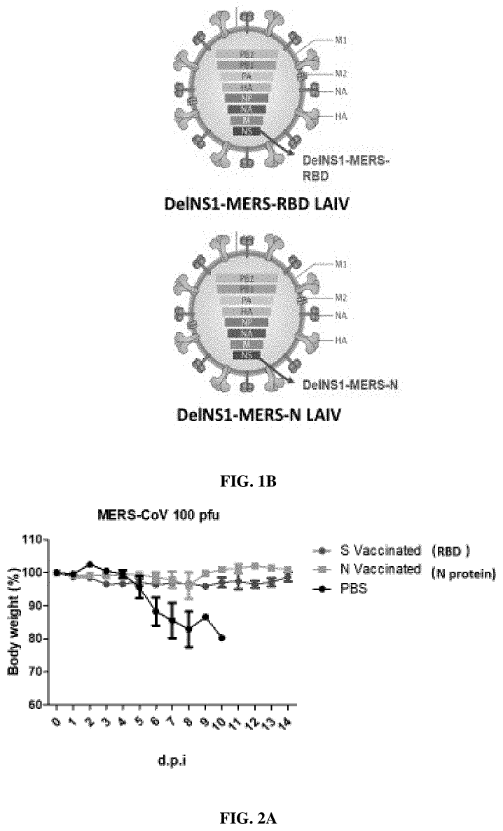

Transgenic mice expressing human DPP4 receptor were prime immunized with DelNS1-MERS-RBD, DelNS1-MERS-N, or control (PBS) twice respectively, four weeks apart Immunized mice were then challenged with lethal dose of MERS coronavirus (100 pfu=2 MLD₅₀). Mice were monitor for 14 days for body weight loss and mortality. The data is shown in FIGS. 2A and 2B.

### Example 3. Protection of DPP4 Transgenic Mice with Inoculation of Lethal Challenge of MERS Coronavirus (10 MLD₅₀)

Transgenic mice expressing human DPP4 receptor were prime immunized with DelNS1-MERS-RBD LAIV, DelNS1-MERS-N LAIV, or DelNS1 LAIV twice respectively, four weeks apart Immunized mice were then challenged with lethal dose of MERS coronavirus (500 pfu=10 MLD₅₀). Mice were monitored for 14 days for body weight loss and mortality.

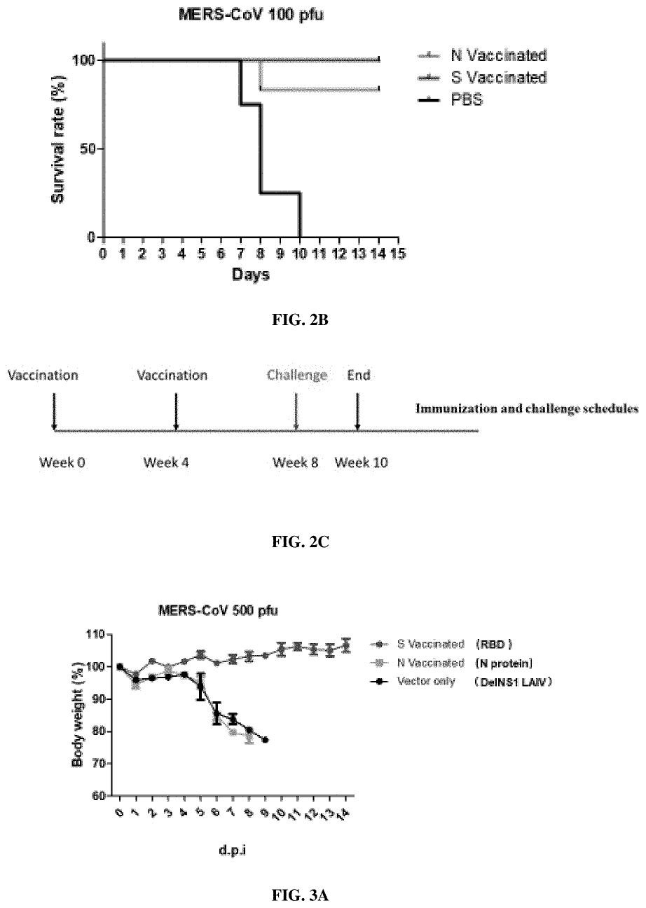

The data is shown in FIGS. 3A and 3B.

### Example 4. Cloning of 2019 Novel Coronavirus (Sars-CoV-2) into DelNS1 LAIV Vector

The sequences for the Receptor Binding Domain of the Sars-CoV-2 is shown in FIG. 4B.

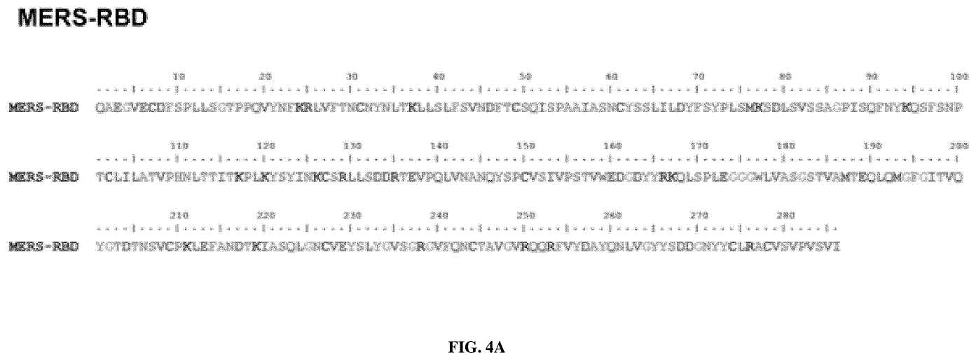

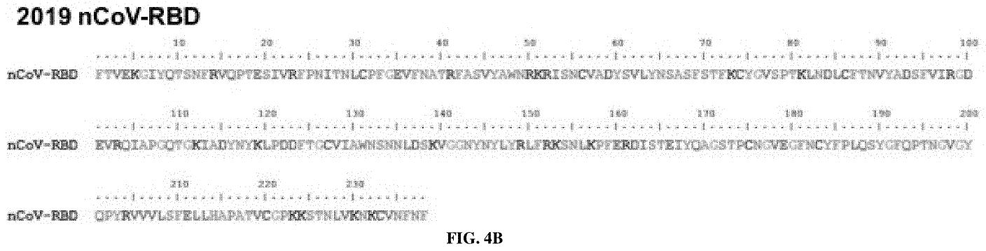

The gene segment containing the RBD from Sars-CoV-2 was cloned into NS segment of CA04-DelNS1 LAIV (Wang et al., mBio 10 (5):e12180-19 (2019)) as depicted in FIG. 5. Verification of NS segment and RBD insert in DelNS1-Sars-CoV-2-RBD vaccine strain is shown in FIG. 6. RNAs were extracted from DelNS1 vaccine strains, CA04-DelNS-Sars-CoV-2-RBD, HK68-DelNS1-Sars-CoV-2-RBD, 4801-DelNS1-Sars-CoV-2-RBD and H1N1 (2019)-DelNS1-Sacs-CoV-2-RBD, after passage in eggs. RT-PCR with primers specific for the NS segment and RBD of Sars-CoV-2 were performed and PCR products were analyzed be agarose electrophoresis. Correct size of PCR products, NS an RBD were observed from all DelNS1 vaccine strains.

### Example 5. Expression of Sars-CoV-2 RBD in DelNS1-Sars-CoV-2-RBD Live Attenuated Virus Infected MDCK Cells

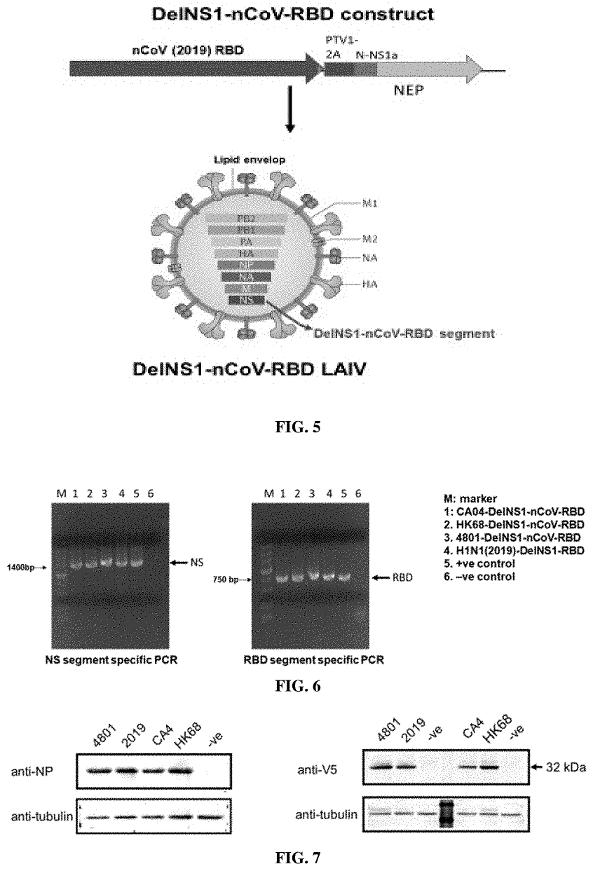

MDCK Cells were infected with CA04-DelNS-Sars-CoV-2-RBD, HK68-DelNS1-Sars-CoV-2-RBD, 4801-DelNS1-Sars-CoV-2-RBD, or H1N1 (2019)-DelNS1-Sars-CoV-2-RBD at 0.1 MOI, or mock infection for 16 hours. Cell lysates were harvested and analyzed by Western blot using either anti-NP (for viral protein NP) or anti-V5 (for RBD which is tagged with a V5 epitope). It is shown that RBD is expressed from all DelNS1 vaccines strains (FIG. 7).

### Example 6. Protection of ACE2 Transgenic Mice from Disease Caused by SARS-CoV-2 Infection

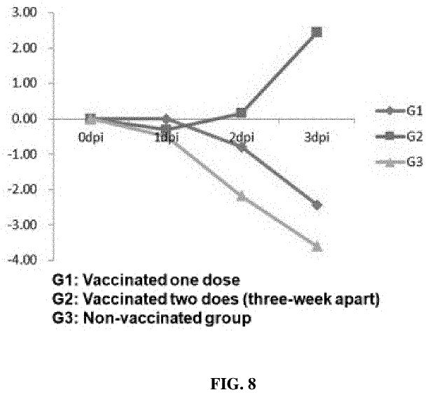

ACE2 transgenic mice were inoculated with CA04-DelNS-Sars-CoV-2-RBD LAIV once or twice (in three-week apart). Three weeks after the last vaccination, mice were challenged with 1×10⁵ TCID₅₀ of SARS-CoV-2 or PBS (control). Mice were observed for body weight change after virus challenge (FIG. 8). Mice immunized with CA04-DelNS-Sacs-CoV-2-RBD LAIV show less body weight loss (one dose) or no body weight loss and gain body weight after three days post infection (two doses).

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “an aptamer” includes a plurality of such aptamers, reference to “the aptamer” is a reference to one or more aptamers and equivalents thereof known to those skilled in the art, and so forth.

Throughout the description and claims of this specification, the word “comprise” and variations of the word, such as “comprising” and “comprises,” means “including but not limited to,” and is not intended to exclude, for example, other additives, components, integers or steps.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Although the description of materials, compositions, components, steps, techniques, etc. may include numerous options and alternatives, this should not be construed as, and is not an admission that, such options and alternatives are equivalent to each other or, in particular, are obvious alternatives. Thus, for example, a list of different moieties does not indicate that the listed moieties are obvious one to the other, nor is it an admission of equivalence or obviousness.

We claim:

1. A live attenuated chimeric virus comprising (a) an influenza virus genome, wherein the influenza virus genome comprises a deletion of a virulence factor activity, and optionally, a first set of one or more mutation(s) that confers replication at 37° C. in the absence of the virulence factor activity; and a second set of one or more mutation(s) that confers replication at a temperature below 35° C., and (b) an insertion of one or more genes encoding one or more Sars-CoV-2 antigens (CoV2Ag).

2. The attenuated chimeric virus of claim 1, wherein the influenza virus genome is from an influenza virus A subtype H1N1 or H3N2.

3. The attenuated chimeric virus of claim 2, wherein the influenza virus genome is from an influenza virus A subtype H1N1 or H3N2 strain selected from the group consisting of CA04 (A/California/04/2009); HK68 (strain A/Hong Kong/1/68), 4801 (H3N2 A/HK/4801/2014), H1N1 (2019); A/WSN/33 and A/PR/8/34.

4. The attenuated chimeric virus of claim 1, wherein the deletion of virulence factor activity comprises a deletion of at least part of a virulence factor gene.

5. The chimeric virus of claim 1, wherein the deletion comprises a deletion of at least part of Non-Structural Protein 1 (NS1) gene extending beyond nucleotides 57 to 528 of an NS1 segment of the mutated virus.

6. The chimeric virus of claim 1, comprising a first set of one or more mutation(s), wherein the first set of one or more mutation(s) comprises a first set of one or more point mutation(s) that confer replicative competence.

7. The chimeric virus of claim 1, wherein the first set of one or more point mutation(s) lies outside of an M region of the mutated influenza virus.

8. The chimeric virus of claim 3, wherein the influenza virus genome is from the A/California/04/2009 influenza strain, and at least one of the first set of one or more point mutation(s) is a G346A mutation in the viral genome.

9. The chimeric virus of claim 1, wherein the virus replicates poorly in MDCK cells at 37° C., when compared to its replication at 33° C. in the MDCK cells.

10. The chimeric virus of claim 1, wherein the second set of one or more mutation(s) comprises a second set of one or more point mutation(s).

11. The chimeric virus of claim 3, wherein the first set of one or more mutation(s) comprises an A14U substitution in the 3′ noncoding region of the M segment of viral RNA.

12. The chimeric virus of claim 1, wherein at least one member of the second set of one or more point mutation(s) is selected from the group consisting of a T261G and an A310G mutation in the influenza virus genome.

13. The chimeric virus of claim 12, comprising a third set of one or more mutation(s) that confers replication at a temperature below 35° C.

14. The chimeric virus of claim 12, wherein the third set of one or more mutation(s) comprises a third set of one or more point mutation(s) that is distinct from the second set of one or more point mutation(s), and is selected from the group consisting of a T261G and an A310G mutation in the H1N1 influenza virus genome.

15. The chimeric virus of claim 1, the antigen is not full length spike protein of Sars-CoV-2, and optionally, wherein the one or more CoV2Ag is the Sar-CoV-2 receptor binding domain (RBD).

16. The chimeric virus of claim 1, selected from the group consisting of CA04-DelNS1-Sars-CoV-2-RBD; HK68-DelNS1-Sars-CoV-2-RBD; 4801-DelNS1-Sars-CoV-2-RBD and H1N1 (2019)-DelNS1-Sars-CoV-2-RBD.

17. A pharmaceutical composition comprising an effective amount of the chimeric virus of claim 1.

18. The composition of claim 17, further comprising an adjuvant.

19. The composition of claim 17, in a form suitable for nasal administration.

20. A method for increasing an immune response to Sars-CoV-2 in a subject in need thereof, comprising administering the composition of claim 1, to the subject.
